(19) United States
(12) Patent Application Publication
(10) Pub. No.: US 2014/0324149 A1
(43) Pub. Date: Oct. 30, 2014

(54) Delivery System With Profiled Sheath Having Balloon-Oriented Position

(71) Applicant: CAPPELLA, INC., AUBURNDALE, MA (US)

(72) Inventors: David Slattery, Kinvara (IE); Damian Kelly, Loughrea (IE); Therese O'Connor, Athenry (IE); Joseph Kerins, Turlough (IE); Michael Gilmore, Loughrea (IE); Mark Steckel, Sharon, MA (US)

(73) Assignee: CAPPELLA, INC., AUBURNDALE, MA (US)

(21) Appl. No.: 14/245,559

(22) Filed: Apr. 4, 2014

Related U.S. Application Data
(63) Continuation of application No. 11/930,634, filed on Oct. 31, 2007, now abandoned.

(51) Int. Cl. A61F 2/958; A61M 25/10

(52) U.S. Cl. CPC A61F 2/958; A61M 25/1029; A61F 2002/9583; A61M 2025/107
USPC 623/1.11; 29/428

(57) ABSTRACT

A delivery system for delivering a self-expanding medical device such as a stent. The delivery system includes a sheath profiled or shaped to reduce instances of interference between a distal edge of the sheath and a vessel or with any lesion or lesions that might be present in the vessel. The sheath is formed by modifying a cylinder of sheath material to include three portions and an initiation slit that controls the rupturing of the sheath to facilitate delivery of the medical device. The initiation slit is positioned on the delivery system with respect to a configuration of a balloon portion of the system.

### RELATED APPLICATIONS

The present application is a continuation of U.S. patent application Ser. No. 11/930,634, filed Oct. 31, 2007, pending, the entire disclosure of which is incorporated herein by reference.

### FIELD OF THE INVENTION

The present invention relates to a delivery system for a self-expanding medical device. More particularly, the delivery system is provided with a profiled sheath allowing for better system positioning of the medical device and which has an initiation slit oriented with respect to a balloon portion of the delivery system.

### BACKGROUND OF THE INVENTION

As is known, treatment of vascular blockages due to any one of a number of conditions, such as arteriosclerosis, often involves balloon dilatation and treatment of the inner vessel wall by placement of a stent. The stent is positioned to prevent restenosis of the vessel walls after the dilatation. Drug eluting stents are now available where medicine is delivered to the vessel wall to also help reduce the occurrence of restenosis.

These stents, i.e., tubular prostheses, typically fall into two general categories of construction. The first category of prosthesis is made from a material that is expandable upon application of a controlled force applied by, for example, a balloon portion of a dilatation catheter upon inflation. The second category of prosthesis is a self-expanding prosthesis formed from, for example, shape memory metals or super-elastic nickel-titanium (NiTi or Nitinol) alloys, that will automatically expand from a compressed or restrained state when the prosthesis is advanced out of a delivery catheter and into the blood vessel.

Some known prosthesis delivery systems for implanting self-expanding stents include an inner lumen upon which the compressed or collapsed prosthesis is mounted and an outer restraining sheath that is initially placed over the compressed prosthesis prior to deployment. When the prosthesis is to be deployed in the body vessel, the outer sheath is moved in relation to the inner lumen to “uncover” the compressed prosthesis, allowing the prosthesis to move to its expanded condition. Some delivery systems utilize a “push-pull” type technique in which the outer sheath is retracted while the inner lumen is pushed forward. Still other systems use an actuating wire that is attached to the outer sheath.

Delivery systems are known where a self-expanding stent is kept in its compressed state by a sheath positioned about the prosthesis. A balloon portion of the delivery catheter is provided to rupture the sheath and, therefore, release the prosthesis. For example, in U.S. Pat. No. 6,656,213, the stent is provided around the balloon, with the sheath around the stent, that is, the balloon, stent, and sheath are co-axially positioned, such that expansion of the balloon helps to expand the self-expanding stent as well as rupture the sheath.

There have been issues, however, with the sheath having an adverse effect on the vessel as the delivery system is positioned. In some instances, the sheath has been “caught” on lesions that are found in the vessel.

There is, therefore, a need for a sheath that does not interfere with positioning of the delivery system.

### SUMMARY OF THE INVENTION

Embodiments of the present invention serve to minimize any adverse effects of the sheath on either the positioning of the delivery system or the vessel itself. The sheath is made with a profile and leading edge that, as the delivery system is distally moved through a vessel, does not interfere with positioning. The profiled sheath also reduces incidences of the sheath catching on lesions in the vessel. Further, an initiation slit provided on the sheath is oriented with respect to a balloon portion of the delivery system.

In one embodiment, a delivery system includes a catheter having a distal end and a proximal end; a balloon positioned at the distal end of the catheter, the balloon comprising at least two wing portions wrapped about the distal end of the catheter; a medical device, having a compressed state and an expanded state, positioned about the balloon portion; and a sheath positioned about the medical device to hold the medical device in the compressed state. The sheath has a distal sheath portion located at a distal end of the sheath, the distal sheath portion having a first diameter and a first longitudinal length; a transition portion, of a second longitudinal length, having a distal end located adjacent the proximal end of the distal sheath portion, the distal end of the transition portion being of the first diameter and having a proximal end with a second diameter greater than the first diameter; a body portion of a third longitudinal length, having a distal end adjacent a proximal end of the transition portion, the body portion being of the second diameter; and an opening provided in an outer surface of the sheath. The opening is located on the positioned sheath in a predetermined relation to the at least two wing portions of the balloon.

The opening of the positioned sheath is located at a position where a total force exerted by expansion of the at least two wing portions against the positioned sheath, upon inflation of the balloon, is at its greatest.

In one embodiment, the balloon is a dual-wing balloon having first and second wings, each wing having a respective wing-tip portion and a wing-base portion, wherein the balloon is wrapped about the catheter in a bi-fold orientation, and wherein the opening in the sheath is located between the wing-tip portion of the first wing and the wing-base portion of the second wing.

In one embodiment, the balloon is a dual-wing balloon having first and second wings, each wing having a respective wing-tip portion and a wing-base portion, and wherein the balloon is wrapped about the catheter in a U-fold orientation, and wherein the opening in the sheath is located between the wing tip of the first wing and the wingtip of the second wing.

In one embodiment, the balloon is a tri-wing balloon having three wings, each wing having a respective wingtip portion and wing base portion, wherein the balloon is wrapped about the catheter such that a wingtip portion of a first wing is folded toward a wing-base portion of a next adjacent wing, and wherein the opening in the sheath is located between the wingtip portion of the first wing and the wing-base portion of the next adjacent wing.

### BRIEF DESCRIPTION OF THE DRAWINGS

The above and further advantages of the present invention may be better understood by referring to the following description in conjunction with the accompanying drawings in which:

FIG. 1 is a representation of a known ostial protection device;

FIG. 2 is a representation of a known device delivery system;

FIG. 3 is a cross-section view of the delivery system of FIG. 2;

FIGS. 4 and 5 represent operation of the delivery system of FIG. 2 in a vessel;

FIG. 6 is a cross-section view of the delivery system as shown in

FIG. 5;

FIG. 7 is a representation of a known sheath;

FIG. 8 is a representation of the sheath of FIG. 7 while being positioned in a vessel;

FIG. 9 is a profiled sheath in accordance with one embodiment of the present invention;

FIG. 10 is a representation of the sheath shown in FIG. 9 positioned on a delivery system;

FIG. 11 is a representation of a portion of the sheath of FIG. 9 as the sheath is being expanded:

FIGS. 12-14 represent one embodiment of a method of manufacturing the sheath of FIG. 9;

FIG. 15 is a flowchart of the steps of an embodiment of a method of manufacturing the sheath of FIG. 9;

FIG. 16 is a perspective view of a dual-wing PTCA balloon;

FIG. 17 is a cross-sectional view of the dual-wing PTCA balloon as shown in FIG. 16;

FIG. 18 is a cross-sectional view of the dual-wing PTCA balloon of FIG. 17 in a bi-folded configuration and wrapped within a sheath;

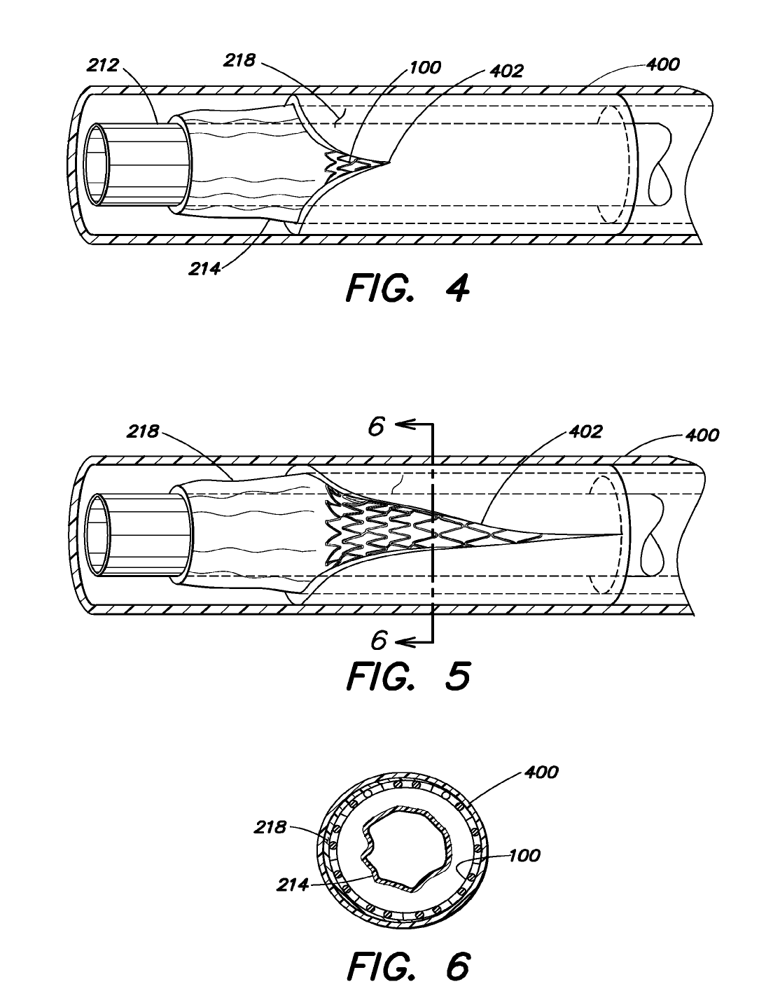

FIG. 19 is a cross-sectional view of the partially expanded PTCA balloon of FIGS. 3 and 4;

FIG. 20 is a cross-sectional view of a tri-wing PTCA balloon;

FIG. 21 is a cross-sectional view of the tri-wing PTCA balloon of FIG. 20 in a tri-folded configuration and wrapped within a sheath;

FIG. 22 is a cross-sectional view of a dual-wing PTCA balloon in a U-fold configuration and wrapped within a sheath;

FIG. 23 is a method of placing a sheath initiation opening with respect to an orientation of a balloon placed within; and

FIG. 24 is an alternate method of loading a device on a delivery system and orienting the sheath initiation opening with a balloon.

### DETAILED DESCRIPTION

The present invention is directed to a sheath that is profiled, i.e., shaped, to reduce instances of interference between a distal edge of the sheath and a vessel and/or any lesion or lesions that might be present in the vessel. Embodiments of the sheath and its implementation will be described below in more detail.

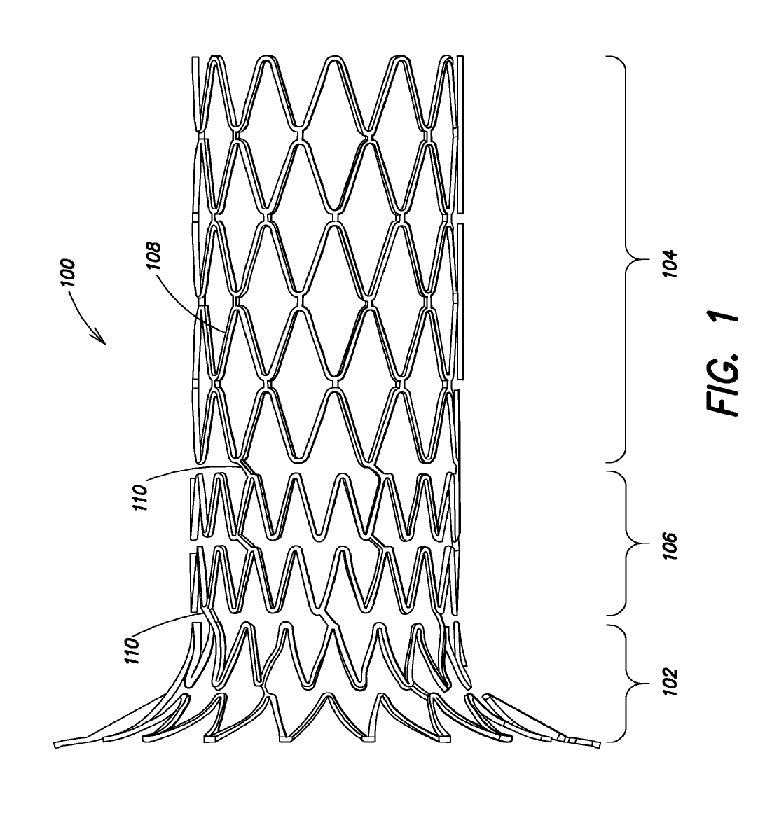

Reference is now made to FIG. 1, which illustrates a schematic view of a device 100, for example, an ostial protection device as described in co-pending US application SN 11/252,224 filed Oct. 17, 2005 for “Segmented Ostial Protection Device,” and which is herein incorporated by reference in its entirety. It should be noted that the present description is with reference to an ostial protection device for purposes of explanation only. The claims are not limited to systems with medical devices intended for insertion at an ostium.

The device 100 includes a cap or flared portion 102, an anchor portion 104, and an articulating portion 106. The anchor portion 104 is configured to fit into a side-branch vessel and the cap portion 102 is configured to selectively protect at least part of an ostial region. The articulating portion 106 flexibly connects the anchor portion 104 to the cap portion 102, such that various angles of articulation are possible between each of the three portions. The articulating portion 106 includes connectors 110 connecting to the cap portion 102 and to the anchor portion 104.

The device 100 may be formed of a generally elastic, super-elastic, in-vivo stable and/or “shape-memorizing” material. Such a material is able to be initially formed in a desired shape, e.g., during an initial procedure performed at a relatively high temperature, deformed, e.g., compressed, and then released to assume the desired shape. The device 100 may be formed of Nickel-Titanium alloy (“Nitinol”) that possesses both super-elastic and shape-memorizing properties. Biocompatible non-elastic materials, such as stainless steel, for example, may be also used. Other combinations of materials and processes would be understood by one of ordinary skill in the art.

The device 100 may be formed from a wire or cut from a single tube of material. The device 100 may be formed from a single piece of material or may be assembled in sections. In general, each section comprises a plurality of struts 108 arranged in a manner of peaks and valleys familiar to those of ordinary skill in the art.

The struts 108 may have a cross-section that is, but not limited to, circular, oval, rectangular, or square. One of ordinary skill in the art will understand the options available with respect to the cross-section chosen for the struts 108 depending upon the intended application of the device.

The self-expanding device 100 may be delivered via a system that uses a sheath and a balloon portion of a delivery catheter. In general, as explained in more detail below, the device 100 is compressed and loaded in a low-profile or crimped state about a balloon portion and surrounded by a sheath. To deliver the device the balloon portion is inflated, causing the sheath to rupture and release the constrained device 100 into its expanded condition within the vessel.

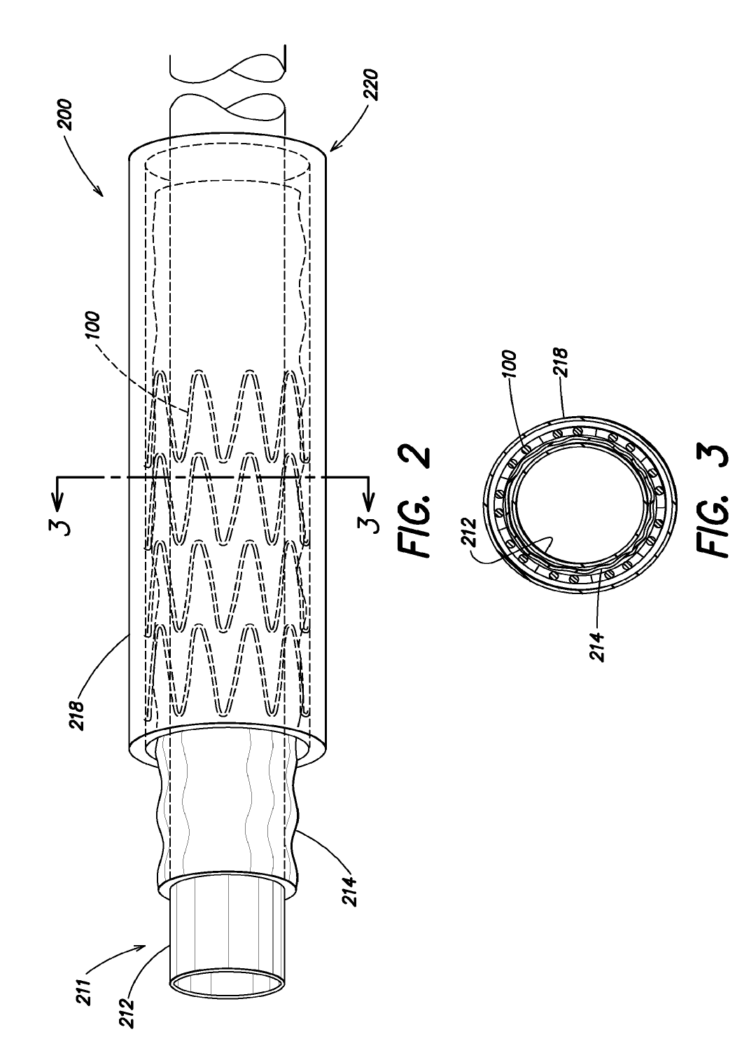

A medical device delivery system 200, as shown in FIG. 2, includes a delivery catheter 212 with a balloon portion 214 positioned at a distal end 211 of the catheter 212. As is known, a lumen is provided to inflate the balloon portion 214 as necessary during the procedure to deliver the device 100 that is placed at the distal end of the catheter 212 and around the balloon portion 214. As per the present discussion, the device 100 is a self expanding device and, therefore, a cylindrical sheath 218 is also disposed at the distal end 211 of the catheter 212 so as to enclose the device 100 and the balloon portion 214. The sheath 218 is attached to the catheter 212 at some point 220 proximal to the distal end 211 of the catheter 212.

A cross-section view of the system 200, along line 3-3, is presented in FIG. 3. As shown, the sheath 218 surrounds the stent or device 100 and the balloon portion 214 positioned on the catheter 212.

The sheath 218 may be made from a material having a grain, or fibers, that can be longitudinally oriented, for example, PTFE, Nylon, PEBAX, polypropylene, and the like. Other materials may be used for the sheath as easily understood by one of ordinary skill in the art.

Referring now to FIG. 4, the delivery system 200 is positioned at a desired location within a vessel 400. The balloon portion 214 is inflated causing the sheath 218 to rupture. As the sheath 218 ruptures, the device 100 is released to expand within the vessel 400. The sheath 218 will rupture or split, as shown in FIG. 5, and due to the elastic properties of the sheath 218, will no longer constrain the device 100. In general, the sheath 218, upon expansion of the balloon portion 214, will tear or rupture along a perforation or initial cut 402 in substantially a straight line following a longitudinal axis of the sheath 218 as defined, generally, by the catheter 212.

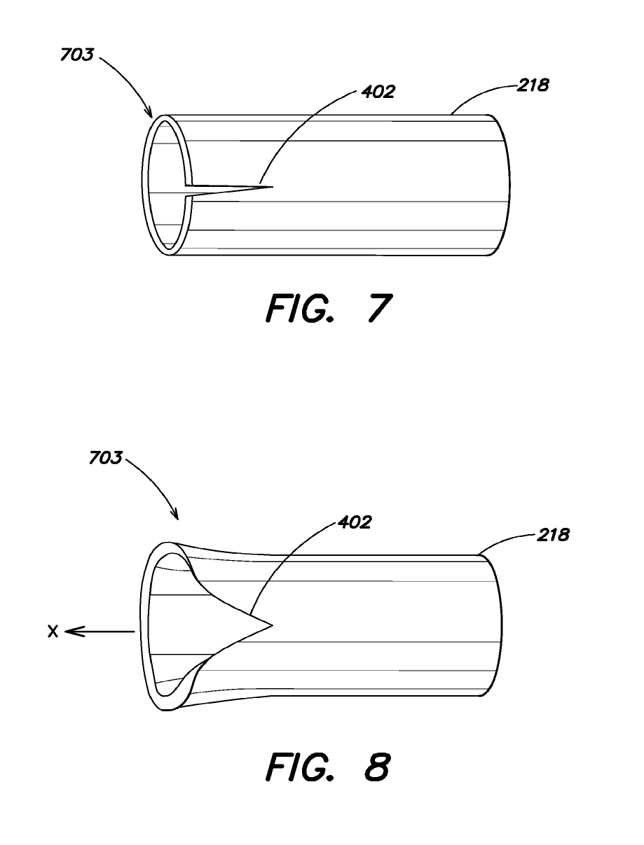

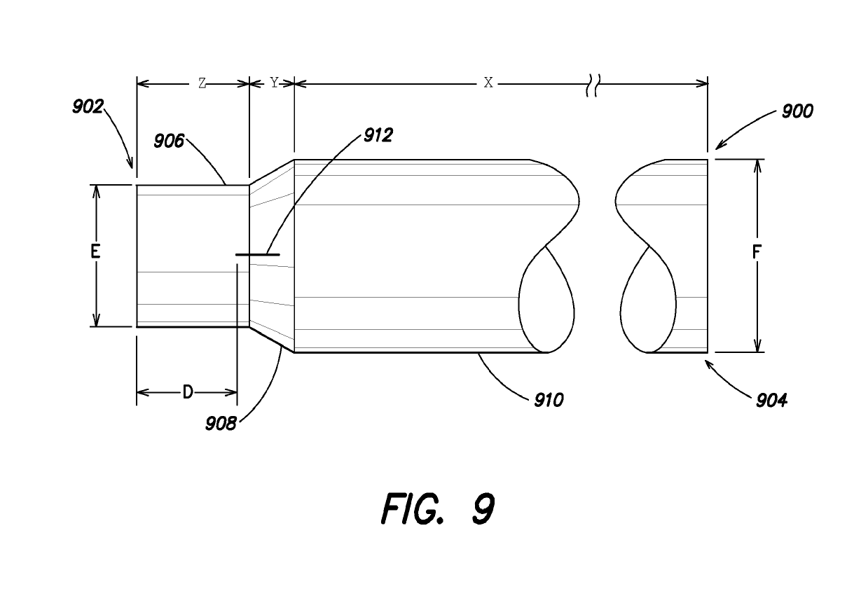

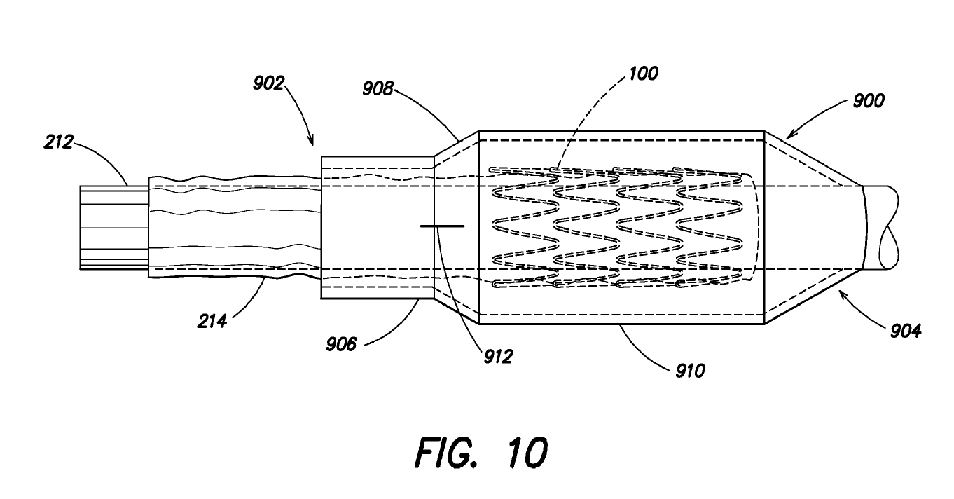

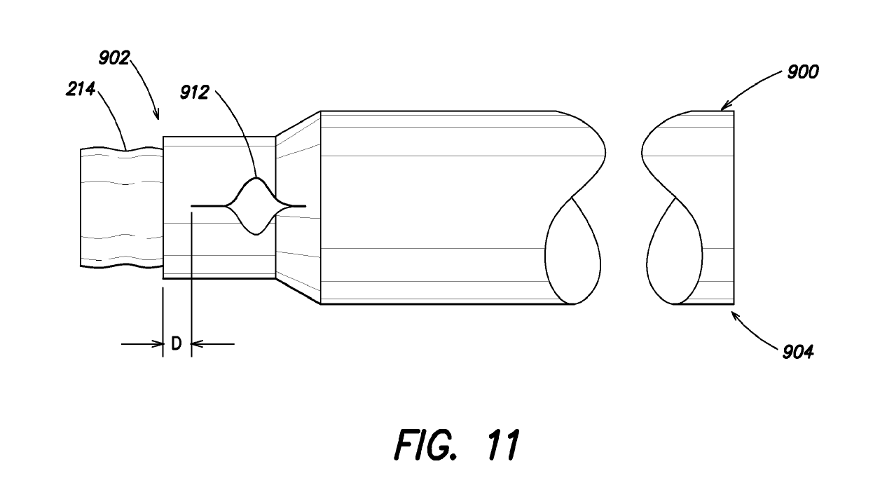

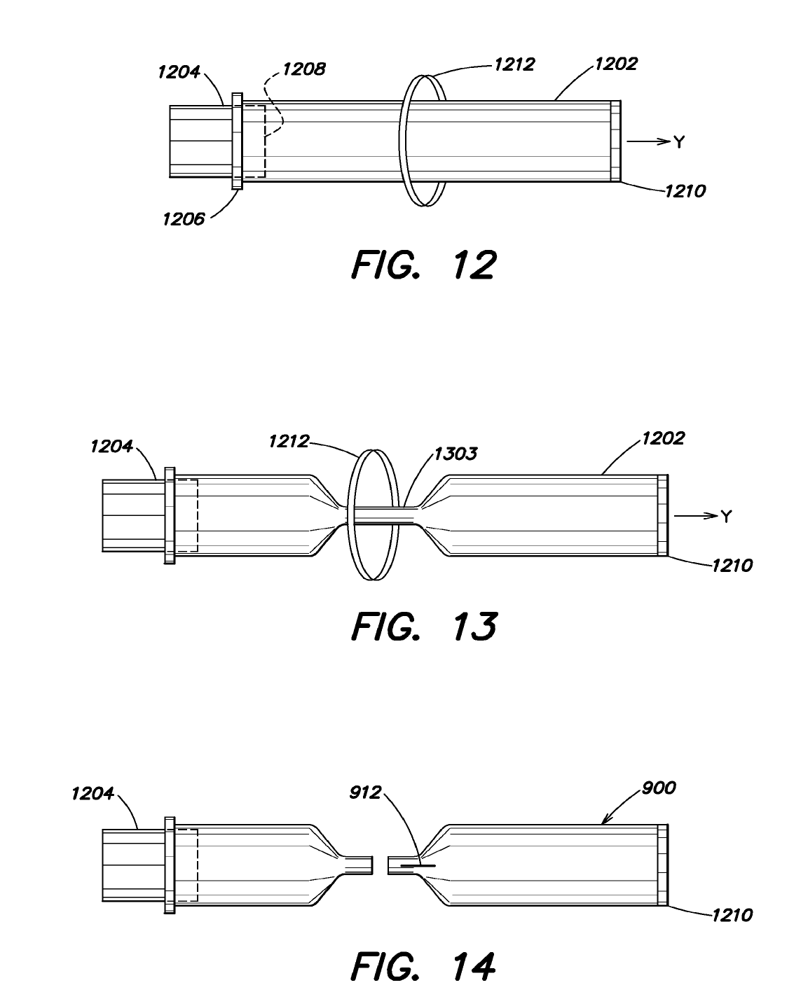

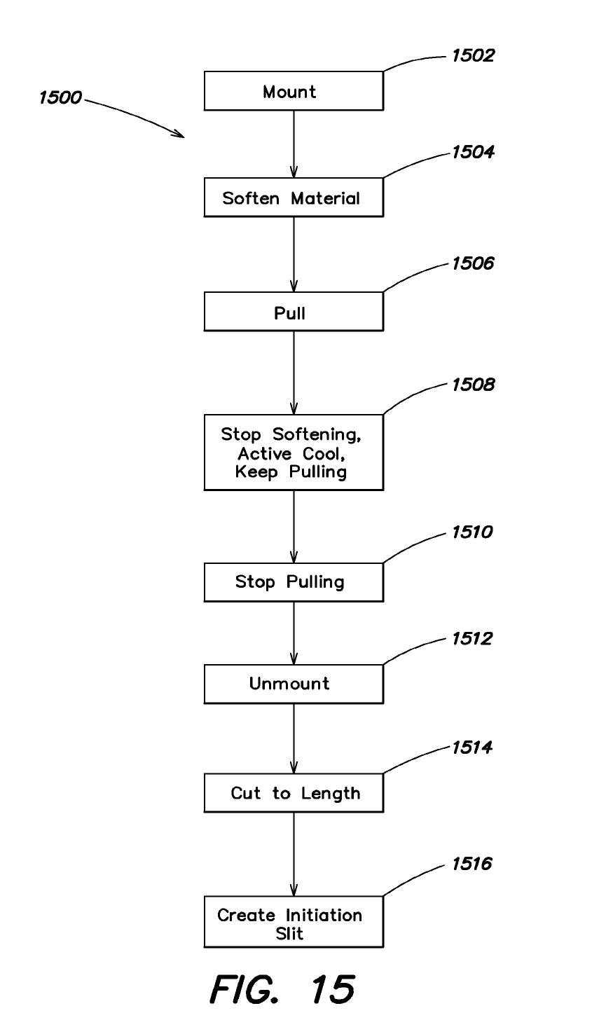

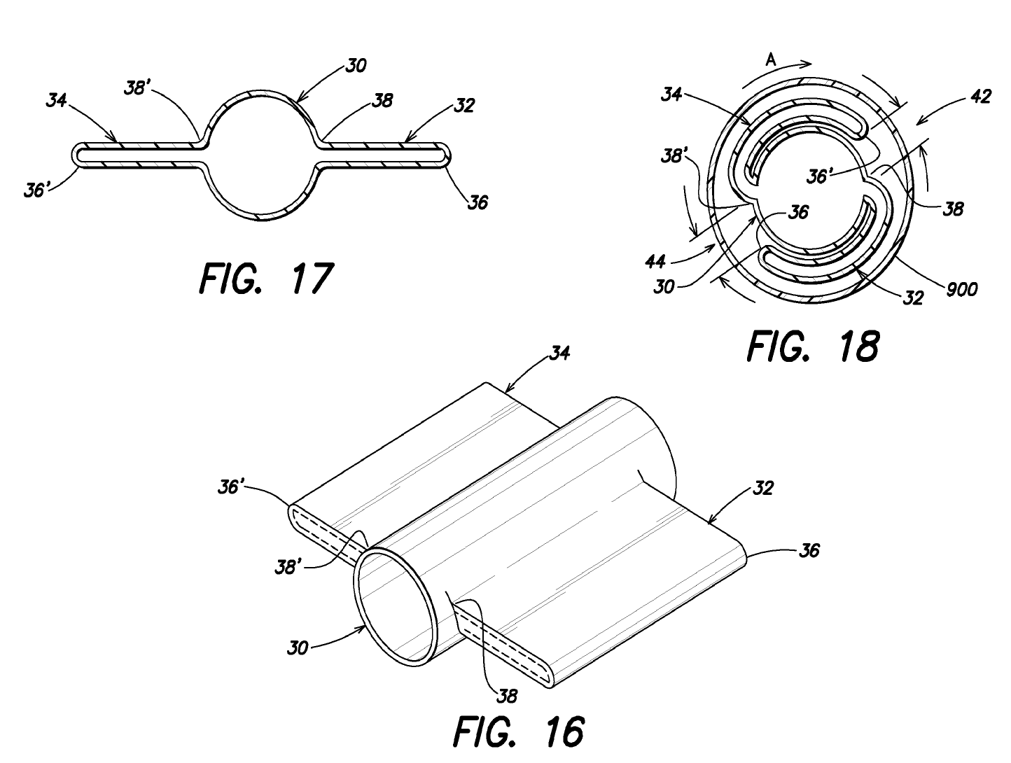

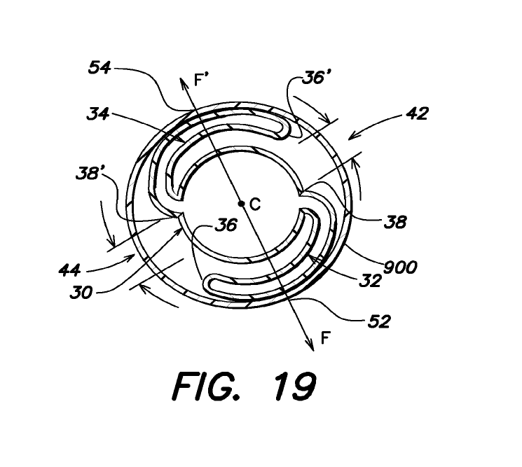

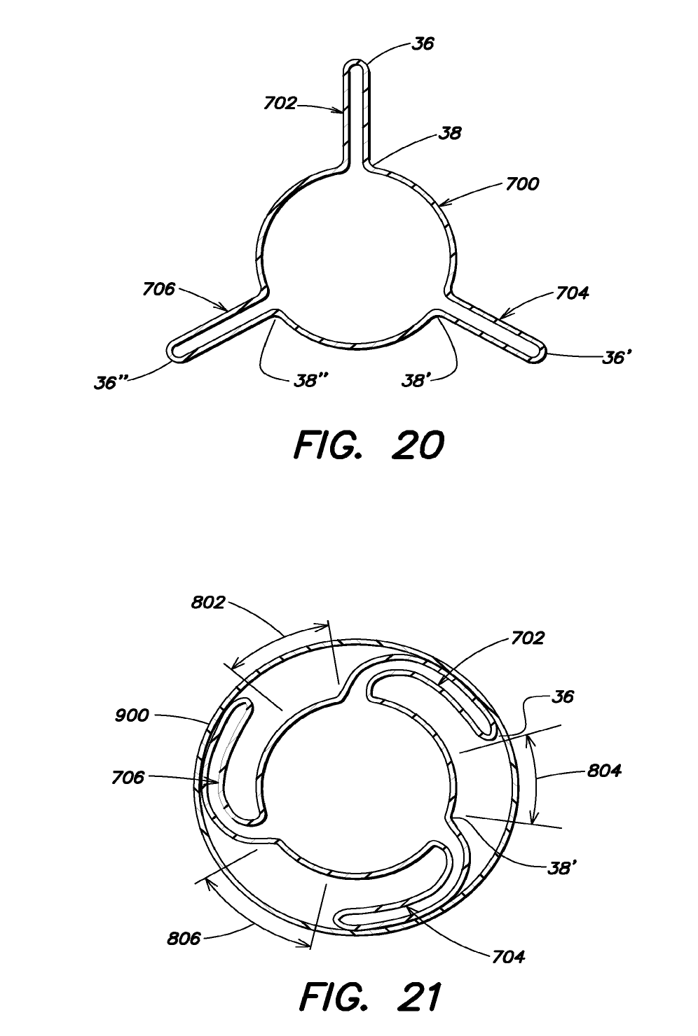

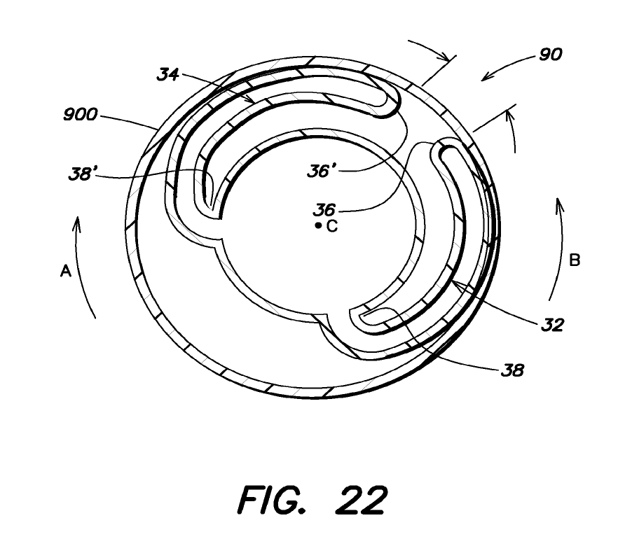

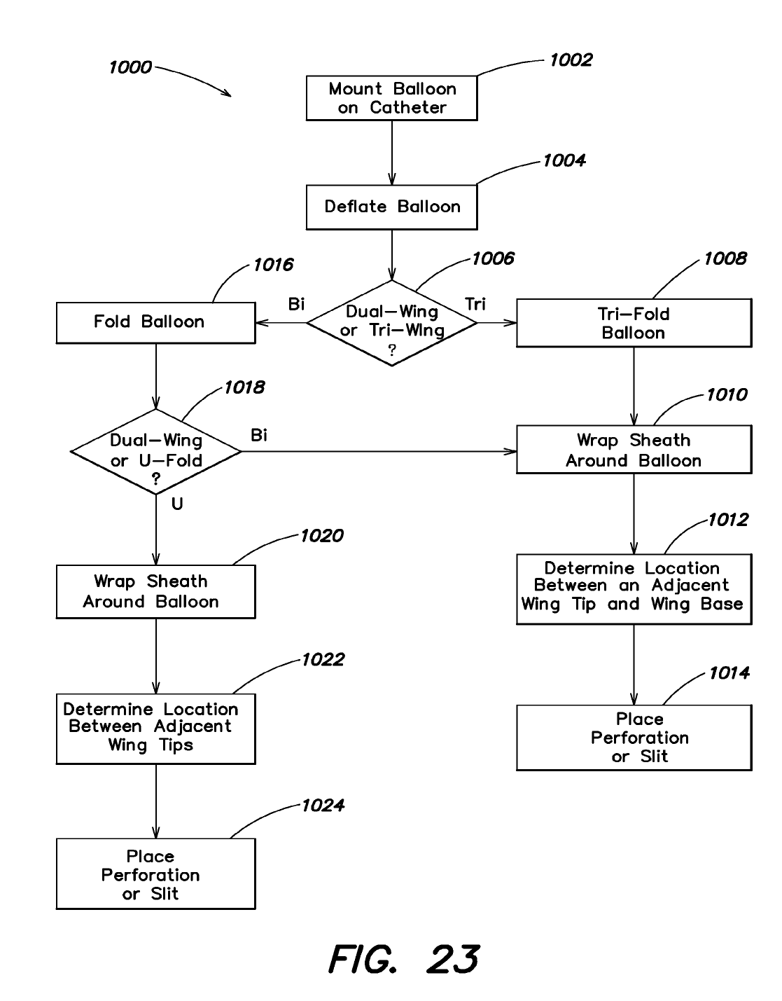

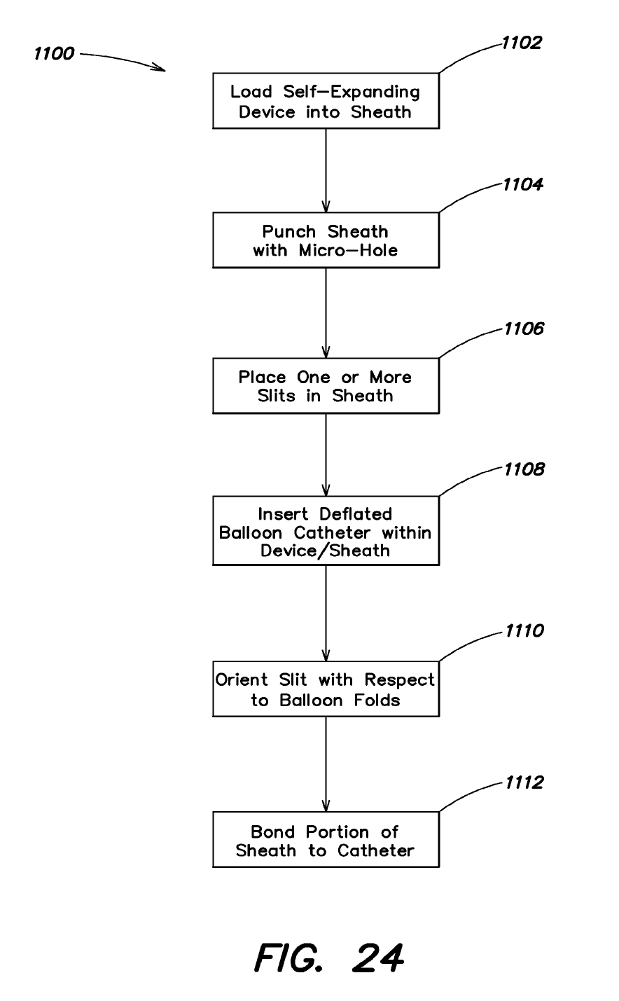

The sheath 218 is made from a plastic material and, as above, is generally cylindrical, i.e., a hollow tube. Once the sheath 218 ruptures, however, it is no longer a cylinder and has a form that covers less than all of the circumference of the now-expanded stent 100. Referring to FIG. 6, a cross-section view of the system 200 of FIG. 5 along the line 6-6, the now-deflated balloon portion 214 is within the lumen of the expanded stent 100. The ruptured sheath 218 is trapped between a portion of the now-expanded stent 100 and the vessel wall 400. The ruptured sheath 218, however, is only trapped between the stent 100 and the vessel wall 400, for a portion, i.e., less than all, of the circumference of the now-expanded stent 100.

Referring now to FIG. 7, the sheath 218, has an initial slit 402 that extends proximally from a distal end 703 of the sheath 218. The representation of this sheath 218, shown in FIG. 7, is its configuration when placed around the balloon portion 214 and stent 100 on the delivery system 212, but prior to inflation of the balloon portion 214.

Turning now to FIG. 8, as the delivery system, not shown, is maneuvered through the vessel in a distal direction X, as shown by the arrow, there have been incidents where the distal end 703 of the sheath 218 begins to spread apart and enlarge. One theory is that as the delivery system is inserted through a curved vasculature, portions of the sheath 218 extend in a direction opposite to that of the curving delivery system. It has been noted that this expansion of the sheath 218 is similar to an open mouth of a fish. This “fish-mouthing” of the sheath 218 is problematic as the sheath 218 may catch on lesions in the vessel and/or prevent the delivery system from properly tracking distally across a lesion. This interference with the tracking, or the catching on lesions, can lead to significant complications in the procedure, and may increase the time of the procedure, any of which can adversely affect patient safety.

The inventors of the present application have noted that adjustment of either the inner diameter of the sheath or the wall thickness of the material from which the sheath is made, does not sufficiently reduce the occurrence of fish-mouth. It appears that the initiation slit 402 is one of the leading factors that contributes to the size of the fish-mouth. In one series of experiments, the length of the initiation slit 402 was reduced from 1.5 mm to 0.5 mm and the amount of fish-mouth width, i.e., diameter, was substantially reduced. While the amount of fish-mouthing was reduced, however, the benefit of a lower and consistent pressure of the balloon portion necessary to consistently open, i.e., rupture, the sheath 218, was negatively affected. Thus, merely reducing the length of the initiation slit 402, while it does reduce the width of the fish-mouth, prevents consistent release of the device at a lower balloon pressure.

A profiled sheath 900, as shown in FIG. 9, reduces lesion crossing issues and, therefore, reduces the occurrences of complications that may adversely affect the proper and safe delivery of a self-expanding medical device. The profiled sheath 900 includes a distal end 902 and a proximal end 904. A distal lead portion 906 is located at the distal end 902 and has a corresponding longitudinal length C. Located proximal to the distal lead portion 906 is a cone/transition portion 908 having a corresponding longitudinal length of B. Located proximal to the cone/transition portion 908 is a body portion 910 having a corresponding longitudinal length A. As shown, the distal lead portion 906 has a corresponding width E and the body portion 910 has a corresponding width F. The cone/transition portion 908 transitions from the width E to the width F as between the distal lead portion 906 and the body portion 910, respectively.

In the present description, reference to “width” is referring to the diameter of the tubular sheath. Further, while there is reference to “portions,” e.g., distal lead portion 906, the profiled sheath 900 is, in one embodiment, of a unitary construction. The claims appended hereto, however, should not be limited to this construction unless expressly recited therein.

The sheath 900 is made from material similar to that referenced above with respect to the known sheath 218. Further, the grain direction of this material is oriented in a longitudinal direction along the profiled sheath 900 running from the distal end 902 to the proximal end 904.

An initiation opening 912 is provided across a junction or boundary between the distal lead portion 906 and the cone/transition portion 908. A distal-most part of the opening 912 is located a distance D from the distal end 902 of the sheath 900. Thus, the opening 912 is “set back” from the distal end 902 of the sheath 900. It is advantageous to position the opening 912 across the boundary between the distal lead portion 906 and the cone/transition portion 908. The opening 912 need not, however, be symmetrically positioned across the boundary.

The opening 912 may be implemented as a slice in the sheath material, where no material has been removed, and where there are sharp edges at each end of the opening 912. The sharp edges assist in the consistent splitting of the sheath. The opening 912 may be created by a slicing operation or a punching operation. The opening 912 may be implemented by operation of a sharp blade or a slicing laser device could be used. Alternatively, the opening 912 may result from an operation where material is removed, i.e., “punched out.”

Referring now to FIG. 10, the profiled sheath 900 is disposed about a self-expanding stent 100 and a balloon portion 214 of a delivery system 212 in substantially the same way as has been described above with respect to FIG. 2. As shown, the distal end 902 is located about the balloon portion 214, i.e., proximal to a distal end of the balloon portion 214. The sheath 900 is positioned with respect to the stent 100 such that the distal lead portion 906 and the cone/transition portion 908 are located distal to a distal-most end of the device 100. In other words, the device 100 corresponds substantially with the body portion 910 of the profiled sheath 900. Similar to the description above, the proximal end 904 of the profiled sheath 900 is attached to the delivery catheter 212 to facilitate withdrawal of the ruptured sheath subsequent to deployment of the medical device 100.

In operation, as the balloon portion 214 is inflated, the opening 912 will expand as shown in FIG. 11. The fibers of the material from which the profiled sheath 900 is made are oriented longitudinally, therefore, as the balloon portion 214 inflates, the opening 912 will expand and the profiled sheath 900 will rupture. The distance D is chosen to minimize the amount of rupturing of the profiled sheath 900 at the opening 912 due to tensile forces. In one embodiment, an opening 912 approximately 1.5 mm long is placed not more than about 0.5 mm from the distal end 902 of the sheath 900.

A profiled sheath 900 is made from any suitable material for a sheath as has been described above. To make the profiled sheath, the material is initially provided as a cylindrical tube of material 1202 and is attached to one end of a mandrel 1204, as shown in FIG. 12. The material tube 1202 may be attached to a cap portion consisting of a ring 1206 and an inset portion 1208. A coupling ring or tab 1210 is attached to the free end of the material tube 1202. An RF coil 1212 is then positioned about the material tube 1202.

The RF coil 1212 is activated while at the same time a pulling force is applied to the free end 1210 in a direction Y, as shown in FIGS. 12 and 13. The active RF coil 1212 raises the temperature of the sheath material making it soft and malleable. In one embodiment, raising the temperature of the PTFE material to about 230° C. is sufficient to soften the material without melting. The activation of the RF coil 1212, in conjunction with the pulling force on the material 1202, causes the tube material to, generally, create a lengthened portion 1302 of the tube having a smaller diameter than the cylindrical tube initially possessed.

Once the desired profile has been obtained, the narrowed portion of the tube is then cut and the opening 912 is created, as shown in FIG. 14. When the tab portion 1210 is removed, the profiled sheath 900 remains.

As shown in the flowchart of FIG. 15, a method 1500 for manufacturing the profiled sheath 900, with respect to FIGS. 12-14, in accordance with one embodiment of the present invention, begins with mounting the cylindrical material on the mandrel 1204, step 1502. Subsequently, step 1504, the sheath material is softened, e.g., via the RF coil, while the sheath material is pulled, step 1506. In one embodiment, an amount of pressure used to grip the sheath is 6 bar while power to the RF coil is on for approximately 3 seconds. The heating is stopped, i.e., the RF coil is turned off, and the sheath is actively air-cooled with ambient air for about 8 seconds in one embodiment while still maintaining a pulling force on the material, step 1508. In one embodiment, the pull rate is approximately 2.3 mm/sec. After a predetermined amount of time, the pulling is stopped, step 1510, and the material is removed from the mandrel, step 1512. In one embodiment, a stretched length is approximately 5.5 mm. The profiled sheath 900 is then cut to length, step 1514, and the initiation opening is created, step 1516.

While an RF coil has been described for softening the sheath material, a heater, microwave device, steam device, or infrared (IR) laser could also be used. Choosing the apparatus or method for softening the sheath material is within the capabilities of one of ordinary skill in the art.

The effectiveness of the sheath for delivery of a device will be significantly reduced if the delivery system requires too wide a range of balloon pressure to fully split the polymer sheath. The wide range of balloon pressure values required to fully split the sheath renders a system as being too variable to validate and subsequently too variable to use in everyday procedures.

The present inventors have recognized that the bi-folded wings of a PTCA catheter balloon could be used to aid in better controlling the splitting dynamics of the sheath. For reference, a deflated PTCA catheter balloon 30 is shown in a perspective view in FIG. 16 and in cross-section in FIG. 17. The balloon 30 includes, when the PTCA balloon 30 is vacuumed, two substantially equal wings 32, 34. Each wing has a wing tip 36 and a wing base 38.

Referring to FIG. 18, the PTCA balloon 30, once mounted on the delivery system, is folded such that the wings 32, 34 “wrap-around” the body of the balloon 30 in such a way so as to not interfere with each other as the balloon 30 is inflated, i.e., a “wrap bi-fold” orientation. In general, a wing tip portion 36′ of the wing 34 is folded along a circumferential direction A (shown by arrow) toward the base portion 38 of the wing 32. Similarly, the wing tip portion 36 of the wing 32 is folded toward the wing base portion 38′ of the wing 34, continuing in the direction A. Looking along the axis of the system, as shown in FIG. 18, the results of the folds of the balloon in this fashion are similar to a child's pinwheel. A sheath 900, in accordance with an embodiment of the present invention, is then provided over the folded balloon, and the device 100 (not shown) to keep the device 100 in a compressed state.

The placement of the initiation opening 912 to take advantage of the mechanical leverage provided from the folded wings 32, 34 of the balloon 30 will aid in establishing a consistent and repeatable splitting of the sheath at a specific pressure, or relatively narrow range of pressures, of the balloon. In known systems, the split or perforation on the sheath were randomly placed, irrespective of any geometry of the balloon around which the sheath was disposed.

There is an optimum area or areas on the circumference of the sheath at which to place the initiation opening 912 (running longitudinally. These locations around the circumference are determined by the folded balloon.

Referring to FIG. 18, a sheath 900 has been provided around a dual-wing balloon 30 in a wrap bi-folded configuration. Two placement areas 42, 44 along the circumference of the sheath 900 are defined. Placing the initiation opening 912 within at least one of these placement areas optimizes the tearing or rupturing of the sheath 900. These two areas 42, 44 are defined or predetermined with respect to the orientation of the folded balloon.

When the initiation opening 912 is placed anywhere within one of the areas 42, 44, the sheath 900 will split at a uniform and consistent and repeatable pressure of the balloon. It should be noted that one initial cut or perforation in either of the areas 42, 44 is sufficient to initiate the full split of the sheath 900. It has been observed, however, that a split or perforation may be placed in each of the areas 42, 44 to facilitate rupture or separation of the sheath 900.

The specific placement of the initiation opening 912 with respect to the folded geometry or orientation of the balloon provides consistent and repeatable sheath splitting performance. The repeatability and consistency of obtaining a full split provides an advantage with respect to using a delivery system with a balloon expandable sheath to deliver a self expanding medical device.

Thus, the folds or wings 32, 34 of the PTCA balloon 30 play a role in splitting the sheath 900, due to the placement of the initiation opening 912. Further, optimum positions about the circumference of the sheath can be predetermined as a function of the balloon's placement and folded geometry about the catheter.

Referring to FIG. 19, the placement areas 42, 44 can be defined as those locations around the circumference of the sheath 900 at which the resultant force exerted by the wings 32, 34, against the sheath as the balloon is inflated, is at a maximum. It can be considered that the balloon 30 expands symmetrically from its center C as it is being inflated. The wings 32, 34 exert, respectively, forces F and F′, against the sheath 900 at points 52, 54, respectively. The cumulative effect of the forces of the wings 32, 34 against the sheath 900 is maximized in the two placement areas 42, 44. Placing an initiation opening in either or both of the placement areas 42, 44 provides for a repeatable and consistent splitting of the sheath 900 at a known pressure.

The placement areas 42, 44 located about the circumference of the sheath 900 may be considered to be defined as located generally halfway between circumferentially adjacent points where the balloon wings 32, 34 exert a respective force against the sheath 900 upon inflation of the balloon. The placement areas 42, 44, in one embodiment, are located along the circumference of the sheath within a portion of the circumference that is in a range of 40-60% of the distance between the points 52, 54.

Alternatively, the location of the placement areas 42, 44 may be described as being located between a wing tip 36 and a wing base 38 of adjacent wings of the balloon. As shown in FIG. 19, due to the bi-fold of the balloon 30, the wing tip portion 36′ is adjacent the wing base portion 38. The placement area 42 is, therefore, located substantially half-way between these two wing portions. Advantageously, the placement areas 42, 44 are easily discernible by viewing the folded balloon within the sheath.

The balloon 30, as shown in FIG. 17, is of a dual-wing design. Alternatively, a balloon 700 of a tri-wing design, as shown in cross-section in FIG. 20, may be used. As shown, the balloon 700 has three wings 702, 704, 706 symmetrically disposed about the circumference of the balloon. Each of the wings has a wing tip 36 and a wing base 38.

When folded, and placed within a sheath 900, as shown in cross-section in FIG. 21, placement areas 802, 804, 806 are positioned about the circumference of the sheath 900. Similar to the foregoing description, the placement areas 804, 806 are, respectively, located between adjacent wing tip portions 36 and wing base portions 38.

In yet another embodiment, as shown in FIG. 22, the dual-wing balloon is folded in a U-fold, where the wings 32, 34 have their respective wingtip portions 36, 36′ adjacent one another. In this configuration, the wing 34 is wrapped in the circumferential direction A (as shown by the arrow A) while the wing 32 is wrapped in an opposite circumferential direction B (as shown by the arrow B) opposite that of direction A. The placement area 90 is then located along the circumference of the sheath 900 substantially midway between the wingtip portions 36, 36′. It is expected that as the balloon is inflated in this orientation the cumulative effect of the wing portions pushing on this sheath will be maximized within the placement area 90.

A method 1000 for assembling a delivery system as described above is shown, generally, in FIG. 23. Initially, step 1002, the balloon is mounted on the catheter. For the sake of simplicity, reference to a medical device being mounted is not included in this description, however, one of ordinary skill in the art will understand where the medical device would be installed. Subsequently, step 1004, the balloon is mostly deflated, i.e., a vacuum is created within the balloon lumen. At step 1006 it has to be determined whether or not the balloon is of a dual-wing or tri-wing construction. If it is the latter, control passes to step 1008 where the balloon is folded in a tri-fold configuration. The sheath is then wrapped around the balloon and the sheath is bonded to the catheter, step 1010. One or more locations between an adjacent wing-tip and wing-base are then determined at step 1012. Once the location of the placement area is determined in step 1012, the slit is provided at step 1014.

Returning to step 1006, if the balloon is of a dual-wing construction then control passes to step 1016 where the balloon is folded. At step 1018 it is determined as to whether or not the balloon was folded in a bi-fold configuration or a U-fold configuration. If it is determined that it is the former configuration then control passes to step 1010 and operation continues as described above. If, however, it is the U-fold configuration then, at step 1020, the sheath is wrapped around a balloon. Subsequently, step 1022, the location between adjacent wing tips about the circumference of the sheath is determined. Finally, step 1024, the initiation opening is placed in the determined location.

An alternate method 1100 for assembling a system in accordance with another embodiment of the present invention will now be described with respect to the flowchart shown in FIG. 24. Initially, a self-expanding device, for example, the device 100, is loaded into a sheath, step 1102. A micro-hole is then punched into the sheath, step 1104, in order to facilitate the flow-through of liquid, for example, blood, as may be found in a vessel in which the device will be placed. One or more slits or perforations or holes are placed in the sheath, step 1106. A deflated balloon, with its wings folded in one of the orientations described above, is positioned on a catheter which is then inserted within the device/sheath assembly, step 1108. The previously provided slit or perforation is then oriented with respect to the balloon fold, in accordance with the previously described process, step 1110. Once aligned, a portion of the sheath is bonded to the catheter to maintain this orientation, step 1112.

While an embodiment of the present invention has been described with respect to a bi-folded balloon, the invention is not limited to embodiments with a balloon that only has two wings. The present invention can be implemented with any balloon having two or more wings where the initial cut or perforation are placed in the sheath with respect to those points on the sheath at which the wings of the balloon exert force against the sheath as the balloon is being inflated.

Thus, in accordance with the teachings of the present invention, the placement of an initial cut in a sheath that is provided to constrain a self expanding device, for example, a stent prior to delivery, is determined with respect to a geometry and orientation of a folded balloon around which the sheath is provided.

It is to be understood that the present invention is not limited in its application to the details of construction and the arrangement of the components set forth in the foregoing description or illustrated in the drawings. The invention is capable of other embodiments or of being practiced or carried out in various ways. Specifically, while the foregoing description is with respect to a flared ostial protection device, the profiled sheath described here can equally be applied to systems that deliver other types of devices, e.g., a straight or “non-flared” cylindrical main-branch stent.

Also, it is to be understood that the phraseology and terminology employed herein are for the purpose of description and should not be regarded as limiting.

It is further appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although various exemplary embodiments of the present invention have been disclosed, it will be apparent to those skilled in the art that changes and modifications can be made that will achieve some of the advantages of the invention without departing from the spirit and scope of the invention. It will be apparent to those reasonably skilled in the art that other components performing the same functions may be suitably substituted.

What is claimed is:

1. A delivery system, comprising:
a catheter having a distal end and a proximal end;
a balloon positioned at the distal end of the catheter, the balloon comprising at least two wing portions wrapped in a same circumferential direction about the distal end of the catheter, each of said wing portions having a base portion and a tip portion, the tip portion of a first one of said wing portions being spaced a distance from a base portion of a second one of said wing portions;
a medical device, having a compressed state and an expanded state, positioned about the balloon; and
a sheath positioned about the medical device to hold the medical device in the compressed state, the sheath comprising:
a distal sheath portion located about the balloon at a distal end of the sheath, the distal sheath portion having a first longitudinal length and a substantially constant first outer diameter along said first longitudinal length;
a transition portion, of a second longitudinal length, having a distal end and a proximal end, the distal end being located adjacent a proximal end of the distal sheath portion, the distal end of the transition portion being of the first outer diameter, and the proximal end of the transition portion having a second diameter greater than the first outer diameter;
a body portion of a third longitudinal length, having a distal end adjacent the proximal end of the transition portion, the body portion being of the second diameter; and
an opening provided in an outer surface of the sheath,
wherein the opening is located on the positioned sheath in a predetermined relation to the at least two wing portions of the balloon, the predetermined location being in a circumferential region of the sheath along said distance between the tip portion of said first wing portion and the base portion of said second wing portion.

2. The delivery system of claim 1, wherein:
the opening of the positioned sheath is located at a position where a total force exerted by expansion of the at least two wing portions against the positioned sheath, upon inflation of the balloon, is at its greatest.

3. The delivery system of claim 1, wherein:
the opening of the positioned sheath is located at a position that is approximately equidistant between sequentially adjacent circumferential points where the at least two wings press against the positioned sheath as the balloon is inflated.

4. The delivery system of claim 1, wherein:
upon inflation of the balloon, each wing of the at least two wings presses against the positioned sheath at a respective wing pressure location about the circumference of the sheath; and
the opening of the positioned sheath is located at a position that is approximately half the distance, around the circumference, between adjacent wing pressure locations.

5. The delivery system of claim 1, wherein:
the balloon is a dual-wing balloon having first and second wings, each wing having a respective wing-tip portion and wing-base portion,
wherein the balloon is wrapped about the catheter in a bi-fold orientation, and
wherein the opening in the sheath is located between the wing-tip portion of the first wing and the wing-base portion of the second wing.

6. The delivery system of claim 1, wherein:
the balloon is a tri-wing balloon having three wings, each wing having a respective wingtip portion and wing base portion,
wherein the balloon is wrapped about the catheter such that a wingtip portion of a first wing is folded toward a wing-base portion of a next adjacent wing, and
wherein the opening in the sheath is located between the wingtip portion of the first wing and the wing-base portion of the next adjacent wing.

7. The delivery system of claim 1, wherein the sheath comprises:
material with a grain oriented along a longitudinal axis of the sheath, and
wherein the opening is an initiation slit of a predetermined length oriented substantially in parallel with the material grain.

8. The delivery system of claim 7, wherein the slit extends from the distal sheath portion to the transition portion.

9. The delivery system of claim 8, wherein a distal-most end of the initiation slit is located at a predetermined distance proximally from the distal end of the sheath.

10. A method of creating a medical device delivery system, the method comprising:
providing a catheter having a distal end and a proximal end;
wrapping at least two wing portions of a balloon in a same circumferential direction about the distal end of the catheter, each of said wing portions having a base portion and a tip portion, the tip portion of a first one of said wing portions being spaced a distance from a base portion of a second one of said wing portions;
positioning a medical device about the balloon, the medical device configurable in one of: a compressed state and an expanded state; and
providing a sheath about the medical device to hold the medical device in the compressed state about the folded balloon, wherein providing the sheath comprises:
providing a distal sheath portion about the balloon at a distal end of the sheath, wherein the distal sheath portion has a first longitudinal length and a substantially constant first outer diameter along said first longitudinal length;
providing a transition portion, of a second longitudinal length, having a distal end and a proximal end, the distal end being located adjacent a proximal end of the distal sheath portion, the distal end of the transition portion being of the first outer diameter, and the proximal end of the transition portion having a second diameter greater than the first outer diameter;
providing a body portion, of a third longitudinal length, having a distal end adjacent a proximal end of the transition portion, the body portion being of the second diameter; and
providing an opening in an outer surface of the sheath such that said opening extends across a junction between the distal sheath portion and the transition portion; and
locating the opening in the outer surface of the positioned sheath at a location in a predetermined relation to the at least two wing portions of the balloon, the predetermined location being in a circumferential region of the sheath along said distance between the tip portion of said first wing portion and the base portion of said second wing portion.

11. The method of claim 10, further comprising:
positioning the opening of the positioned sheath at a location where a total force exerted by expansion of the at least two wing portions of the balloon against the positioned sheath, upon inflation of the balloon, is at its greatest.

12. The method of claim 10, further comprising:
positioning the opening of the positioned sheath at a location that is approximately equidistant between sequentially adjacent circumferential points where the at least two wing portions press against the positioned sheath as the balloon is inflated.

13. The method of claim 10, wherein
the balloon is a dual-wing balloon having first and second wings, each wing having a respective wing-tip portion and wing-base portion, the method further comprising:
wrapping the balloon about the catheter in a bi-fold orientation, and positioning the opening in the sheath between the wing-tip portion of the first wing and the wing-base portion of the second wing.
